(19) United States
(12) Patent Application Publication
(10) Pub. No.: US 2002/0107184 A1
(43) Pub. Date: Aug. 8, 2002

(54) METHOD FOR TREATING MELANOMA

(76) Inventor: SHALOM Z. HIRSCHMAN, RIVERDALE, NY (US)

Correspondence Address: MYRON COHEN, COHEN PONTANI LIEBERMAN & PAVANE, 551 FIFTH AVENUE SUITE 1210, NEW YORK, NY, 10176

(21) Appl. No.: 08/964,250

(22) Filed: Nov. 4, 1997

(51) Int. Cl. A61K 38/00

(52) U.S. Cl. 514/12

(57) ABSTRACT

The present invention discloses a method for treating patients having melanoma or melanoma associated symptoms by parenterally administering Product R, a peptide-nucleic acid preparation.

### BACKGROUND OF THE INVENTION

1. Field of the Invention

The present invention relates to a method for using Product R as hereinafter defined to treat patients having melanoma.

2. Description of the Related Art

Human melanoma, a malignant melanocytic tumor arising in a pigmented area: skin, mucous membranes, eyes, and CNS, represents the principal cause of death in patients with skin cancer in the United States and Europe. Malignant melanomas vary in size, shape, and color and in their propensity to invade and metastasize. About 40 to 50% of malignant melanomas develop from pigmented moles. Malignant transformation of pigmented moles may result from changes in size or color, especially spread of red, white, and blue pigmentation to surrounding normal skin; changes in surface characteristics, consistency or shape; or signs of inflammation in surrounding skin.

Melanoma includes four major types: lentigo-maligna melanoma which appears on the face or other sun-exposed areas in elderly patients as an asymptomatic, large, flat, tan or brown macule with darker brown or black spots scattered irregularly on its surface; superficial spreading melanoma which is usually asymptomatic and occurs most commonly on women's legs and men's torsos as a plaque with raised, indurated edges, and often shows red, white, and blue spots or small, sometimes protuberant, blue-black nodules; nodular melanoma which may occur anywhere on the body and is seen as dark, protuberant papules or a plaque that varies in color from pearl to gray to black; acroletiginous melanoma which is uncommon and arises on palmar, plantar, and subungual skin.

Melanomas are usually treated by surgical excision, while patients with thick melanomas and those with regional or distant metastasis may benefit from other forms of therapy. Chemotherapy with dacabazine (DTIC) or the nitrosoureas (BCNU,CCNU) are being used with limited success. Cisplatinum and other drugs are currently under study. While using BCG vaccine to alter the patient's immune response have been discouraging, newer forms of immunotherapy such as using interleukin-2 and lymphokine-activated killer cells are promising. Cytokines have been tested in the treatment of different skin cancers during the last decade, and treatment schedules have been established or proposed for several malignant skin tumors. Preferentially, the interferons and interleukin-2 were found to be effective in treating skin cancers including melanoma.

Product R¹ emerged as an antiviral product in the 1930's. While it was originally believed to be a product composed of peptone, peptides and nucleic acids (fully defined hereafter), the precise composition remains unidentified. Nevertheless, Product R has demonstrated an ability to inhibit rapidly the course of several viral diseases. It is nontoxic, miscible with tissue fluids and blood sera and free from anaphylactogenic properties. Interestingly, Hirschman and Chem (J. Invest. Med 1996, 44:347-351) have reported that ¹. The agent is known under the trademark “Reticulose”, a trademark of Advanced Viral Research Corp. Product R stimulates the synthesis of the mRNAs of interferon gamma, interleukin-6 and tumor necrosis factor-alpha, suggesting that Product R may serve as an immune system modulator.

Insofar as the applicant knows, Product R has never been used, nor suggested for treating patients having skin cancer. It is now discovered that Product R is useful in treating patients having skin cancer such as melanoma.

### SUMMARY OF THE INVENTION

The object of this invention therefore is to provide a method for treating patients having melanoma by administering parenterally to the patients Product R, an antiviral agent composed of peptides and nucleic acids.

More specifically, the present invention relates to a method for treating the identified patients by administering parenterally to the patients an effective melanoma treatment amount of Product R from about 5 microliters to about 40 microliters per kilogram of body weight per day in a sterile injectable formulation.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims.

### DETAILED DESCRIPTION OF THE PRESENTLY PREFERRED EMBODIMENTS

As used herein, Product R is the product produced according to either of the following methods.

### Method I For Preparing Product R

Suspend about 35.0 g of casein, about 17.1 g of beef peptone, about 22.0 g of nucleic acid (RNA), about 3.25 g bovine serum albumin in about 2.5 liters of water for injection USP at about 3 to 7° C. in a suitable container and gently stir until all the ingredients have been properly wet. Carefully add while stirring about 16.5 g of sodium hydroxide (reagent grade ACS) and continue stirring until sodium hydroxide completely dissolved. Autoclave at about 9 lbs pressure and 200-230° F. for a period of time until RNA is completely digested, for example, about 4 hours. At the end of the period, the autoclave is stopped and the reaction flask and contents are permitted to slowly cool to ambient temperature. Then cool for at least six hours at about 3-8° C. The resulting solution is filtered through 2 micron and 0.45 micron filters using inert gas such as nitrogen or argon at low pressure (1-6 psi). In a similar manner the solution is filtered again through 0.2 micron pyrogen retention filters. The resulting filtrate is sampled and assayed for total nitrogen. A calculation is then performed to determine the quantity of cooled water for injection to be added to the filtrate to yield a diluted filtrate with a nitrogen content between about 165-210 mg/100 ml, the final volume is approximately 5 liters. The pH is then adjusted with either concentrated HCl (reagent grade ACS) or 1.0 normal NaOH to about 7.3-7.6 range. The diluted solution is then filtered again through 0.2 micron filters with inert gas at low pressure. The final filtrate is then filled and sealed into 2 ml glass ampules while in an inert gas atmosphere. The ampules are collected and autoclaved for final sterilization at 240° F. and 20 to 30 pounds pressure for about 30 minutes. Following the sterilization cycle, the ampules with Product R are cooled and washed.

All quantities are subject to plus or minus 2.5% variation for pH, volume, and analytical adjustments.

### Method II For Preparing Product R

Suspend about 35.0 g of casein, about 17.1 g of beef peptone, about 22.0 g of nucleic acid (RNA), about 3.25 g bovine serum albumin in about 2.5 liters of water for injection USP at about 3 to 7° C. in a suitable container and gently stir until all the ingredients have been properly wet. Slowly add while stirring about 11.75 ml of hydrochloric acid (reagent grade ACS) and continue stirring until hydrochloric acid is completely dissolved. Autoclave at about 9 lbs pressure and 200-230° F. for a period of time until RNA is completely digested, for example, about 4 hours. At the end of the period, the autoclave is stopped and the reaction flask and contents are permitted to slowly cool to ambient temperature. Then cool for at least six hours at about 3-8° C. The resulting solution is filtered through 2 micron and 0.45 micron filters using inert gas such as nitrogen or argon at low pressure (1-6 psi). In a similar manner the solution is filtered again through 0.2 micron pyrogen retention filters. The resulting filtrate is sampled and assayed for total nitrogen. A calculation is then performed to determine the quantity of cooled water for injection to be added to the filtrate to yield a diluted filtrate with a nitrogen content between about 165-210 mg/100 ml, the final volume is approximately 5 liters. The pH is then adjusted with either concentrated HCl (reagent grade ACS) or 35% (w/v) of NaOH to about 7.3-7.6 range. The diluted solution is then filtered again through 0.2 micron filters with inert gas at low pressure. The final filtrate is then filled and sealed into 2 ml glass ampules while in an inert gas atmosphere. The ampules are collected and autoclaved for final sterilization at 240° F. and 20 to 30 pounds pressure for about 30 minutes. Following the sterilization cycle, the ampules with Product R are cooled and washed.

All quantities are subject to plus or minus 2.5% variation for pH, volume, and analytical adjustments.

For patients having all types or stages of melanoma, or melanoma associated symptoms, an effective dose of Product R is in the range of from about 5 microliters to about 40 microliters per kilogram of body weight per day, preferably in the range of about 10 microliters to about 25 microliters per kilogram of body weight per day. Most preferably Product R is administered in an amount of about 30 microliters per kilogram of body weight per day for about one week, followed by about 15 microliters per kilogram of body weight per day in a sterile injectable formulation until the patient becomes asymptomatic. The desired dose may be administered as two, three or more sub-doses at appropriate intervals, generally equally spread in time, throughout the day. Preferably, the full daily dose is administered in one administration.

Preferably, the same dosage as described above may be used to treat patient having Bell's palsy disease inflicted from viral infections.

Product R may be administered by any suitable injection route including, but not limited to intravenously, intraperitoneally, subcutaneously, intramuscularly, and intradermally, etc. The presently preferred route of administration is intramuscularly. It will be appreciated that the preferred route may vary with, for example, the condition and age of the recipient.

Product R may be used in therapy in conjunction with other medicaments including corticosteroid, gamma globulin, glucose, or vitamins, antiviral agents such as interferon or interleukin, etc..

While it is possible for Product R to be administered as part of a pharmaceutical formulation, it is preferable to present it alone, although it may be administered at about the same time as one or more other pharmaceuticals are independently administered. If Product R is administered as part of a pharmaceutical formulation, the formulations of the present invention comprise at least one administered ingredient, as above defined, together with one or more acceptable carriers thereof and optionally other therapeutic ingredients. The carrier(s) must be “acceptable” in the sense of being compatible with the other ingredients of the formulation and not deleterious to the recipient thereof. Preferably, Product R constitutes at least about 90% of such formulation by weight.

The formulations may conveniently be presented in unit-dose or multi-dose containers, e.g. sealed ampules and vials.

Preferred unit dosage formulations are those containing a daily dose or unit, daily sub-dose, or an appropriate fraction of the administered ingredient.

Thus, while there have shown and described and pointed out fundamental novel features of the invention as applied to preferred embodiments thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. It is the intention, therefore, to be limited only as indicated by the scope of the claims appended hereto.

I claim:

1. A method of treating a patient having melanoma associated symptoms, comprising administering parenterally to said patient an effective melanoma treatment amount of Product R in a sterile injectable formulation.

2. The method of claim 1 wherein an effective treatment amount of Product R is in a range from about 5 microliters to about 40 microliters per kilogram of body weight per day in a sterile injectable formulation.

3. The method of claim 1 wherein an effective treatment amount of Product R is in a range from about 10 microliters to about 25 microliters per kilogram of body weight per day in a sterile injectable formulation.

4. The method of claim 1 wherein an effective treatment amount of Product R is about 30 microliters per kilogram of body weight per day in a sterile injectable formulation for about one week, followed by about 15 microliters per kilogram of body weight per day in a sterile injectable formulation until the patient becomes asymptomatic.
